(19) United States
(12) Patent Application Publication
(10) Pub. No.: US 2009/0259149 A1
(43) Pub. Date: Oct. 15, 2009

(54) POWER SUPPLY APPARATUS FOR OPERATION

(76) Inventors: Naoko TAHARA, Hachioji-shi (JP); Koh SHIMIZU, Kodaira-shi (JP)

Correspondence Address: OSTROLENK FABER GERB & SOFFEN, 1180 AVENUE OF THE AMERICAS, NEW YORK, NY, 100368403, US

(21) Appl. No.: 12/103,018

(22) Filed: Apr. 15, 2008

(51) Int. Cl. A61N 7/00

(52) U.S. Cl. 601/2; 606/1

(57) ABSTRACT

A power supply apparatus for operation for outputting power to a surgical instrument includes a resonant frequency detection section for detecting a resonant frequency which minimizes the impedance of the surgical instrument, and an abnormality detection section for detecting whether or not a value of the resonant frequency or a variation amount of the resonant frequency per unit time exceeds a predetermined numerical range or a reference variation value. The predetermined numerical range or the reference variation value is set on the basis of a value and a variation amount of the resonant frequency corresponding to a temperature change of the surgical instrument. By detecting an abnormality in this manner, the surgical instrument can be prevented from being broken.

### BACKGROUND OF THE INVENTION

1. Field of the Invention

The present invention relates to a power supply apparatus for operation.

2. Description of the Related Art

A drive apparatus for an ultrasonic vibrator is hitherto known as a power supply apparatus for operation. For example, in Jpn. Pat. Appln. KOKAI Publication No. 2005-102811, a probe from which a resonant frequency is output by phase-locked loop (PLL) control is described, and in Jpn. Pat. Appln. KOKAI Publication No. 2003-159259, a method for distinguishing breakage of a defective hand-piece in an ultrasonic surgical system and breakage of a defective blade from each other is disclosed. Further, in US2002-0049551, a method for clarifying a difference between a loaded blade and a cracked blade by evaluating a measured impedance difference is disclosed.

### BRIEF SUMMARY OF THE INVENTION

A first aspect of the present invention relates to a power supply apparatus for operation for outputting power to a surgical instrument, the apparatus comprising: a resonant frequency detection section for detecting a resonant frequency which minimizes the impedance of the surgical instrument; a resonant frequency setting section for setting in advance an allowable variation amount of the resonant frequency per unit time as a reference variation amount; and an abnormality detection section for detecting whether or not a detected variation amount of the resonant frequency per unit time exceeds the reference variation amount.

Further, a second aspect of the present invention relates to the first aspect, the power supply apparatus for operation further comprises a temperature detection section for detecting a temperature of the surgical instrument, and the reference variation amount is a variation amount of the resonant frequency which varies in accordance with an amount of change in the temperature of the surgical instrument detected by the temperature detection section.

Further, a third aspect of the present invention relates to the second aspect, in the resonant frequency setting section, an allowable predetermined numerical range of the resonant frequency is further set, and the abnormality detection section further detects whether or not the resonant frequency detected by the resonant frequency detection section is within the predetermined numerical range.

Further, a fourth aspect of the present invention relates to the third aspect, and the abnormality detection section detects whether or not the detected resonant frequency is within the predetermined numerical range of the resonant frequency corresponding to the temperature of the surgical instrument detected in advance.

Further, a fifth aspect of the present invention relates to the fourth aspect, the power supply apparatus for operation further comprises a surgical instrument recognition section for recognizing the type of a connected surgical instrument, and the abnormality detection section detects whether or not the resonant frequency detected by the resonant frequency detection section is within the predetermined numerical range corresponding to the surgical instrument recognized by the surgical instrument recognition section.

Furthermore, a sixth aspect of the present invention relates to the fifth aspect, and when the detected variation amount of the resonant frequency per unit time exceeds the reference variation amount, or when the detected resonant frequency is not within the predetermined numerical range corresponding to the temperature and the type of the surgical instrument, the abnormality detection section stops the power supply to the surgical instrument.

Moreover, a seventh aspect of the present invention relates to the third aspect, and the abnormality detection section detects whether or not the detected resonant frequency is within the predetermined numerical range corresponding to the predetermined temperature change of the surgical instrument.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

FIG. 1 is an external perspective view of an ultrasonic operation system.

FIG. 2 is a view showing a schematic configuration of the ultrasonic operation system.

FIG. 3 is a view showing a state where a drive current generated in an ultrasonic power source unit flows to the hand-piece side.

FIG. 4 is a view showing a relationship between a voltage phase and a current phase.

FIG. 5 is a view for explaining a procedure for scanning for a resonant frequency fr.

(A) in FIG. 6 is a view showing a probe part in an enlarging manner.

(B) and (C) in FIG. 6 are graphs showing frequency dependence of the impedance Z, current I, and phase difference (θV−θI) which are under the PLL control observed when a crack develops in a probe in a normal state.

FIG. 7 is a functional block diagram for explaining a function of each unit in an ultrasonic power source unit in an ultrasonic operation system.

FIG. 8 is a flowchart for detecting an abnormality of a probe according to a first embodiment.

FIG. 9 is a schematic view for explaining a second embodiment, and showing magnitude of each factor in the causation of a variation in a resonant frequency.

FIG. 10 is a flowchart for detecting an abnormality of a probe according to a third embodiment.

FIG. 11 is a functional block diagram for explaining a function of each unit in an ultrasonic power source unit in an ultrasonic operation system according to a fourth embodiment.

FIG. 12 is a flowchart for detecting an abnormality of a probe according to a fifth embodiment.

FIG. 13 is a functional block diagram for explaining a function of each unit in an ultrasonic power source unit in an ultrasonic operation system according to a sixth embodiment.

### DETAILED DESCRIPTION OF THE INVENTION

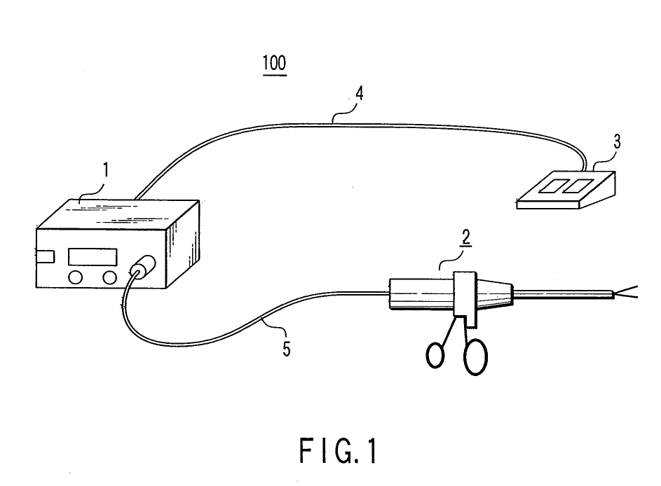

Embodiments of the present invention will be described below in detail with reference to the accompanying drawings. An endoscopic surgical operation for performing medical treatment of a diseased part to be performed by using a scope for observing a state in an abdominal cavity of a patient is known. FIG. 1 is an external perspective view of an ultrasonic operation system used as an example of a system for such an endoscopic surgical operation. The ultrasonic operation system is constituted of an ultrasonic power source unit 1 serving as a power supply apparatus for operation for generating an ultrasonic output for driving an ultrasonic vibrator, a hand-piece 2 serving as an ultrasonic surgical instrument for performing treatment by using an ultrasonic output supplied from the ultrasonic power source unit 1 through a cable 5, and a foot switch 3 connected to the ultrasonic power source unit 1 through a cable 4, for controlling the ultrasonic output from the ultrasonic power source unit 1.

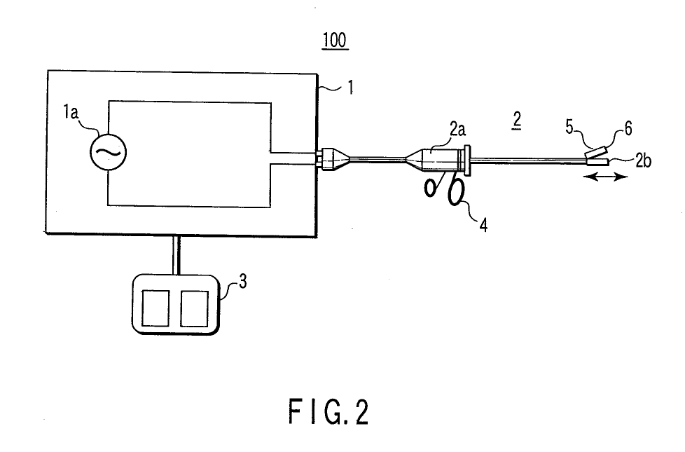

In FIG. 2, the hand-piece 2 is constituted of a hand-piece main body section 2a which includes handles 4, and in which an ultrasonic vibrator (not shown) is incorporated, and a probe 2b for transmitting vibration of the ultrasonic vibrator to a treatment section 5. The ultrasonic power source unit 1 is provided with an ultrasonic oscillator circuit 1a for generating electric energy for vibrating the ultrasonic vibrator. An electric signal output from the ultrasonic power source unit 1 is converted into mechanical vibration (ultrasonic vibration) by the ultrasonic vibrator inside the hand-piece main body section 2a, and thereafter the vibration is transmitted by the probe 2b to the treatment section 5. The treatment section 5 is provided with a grasping section 6 called a jaw driven to be opened or closed with respect to the distal end of the probe 2b. When the handles 4 are operated, the grasping section 6 is driven to be opened or closed with respect to the distal end of the probe 2b, and coagulation or incision of living tissue is performed by utilizing frictional heat generated by holding the living tissue between the distal end of the probe 2b and the grasping section 6 and applying the ultrasonic vibration thereto.

In this probe 2b, a crack is caused due to a scratch received when the probe 2b comes into contact with forceps or a clip during an operation. When a crack is caused to the probe 2b during an operation, it is necessary to immediately stop ultrasonic vibration, and replace the probe with a new one. If the operation is continued in the state where the crack is caused to the probe, it is conceivable that there is the possibility of the probe part being broken and falling off. Accordingly, it becomes necessary to detect the occurrence of the crack at an early stage, and inform the medical pursuer of the occurrence of the crack.

The ultrasonic operation system will be described below in detail, and an apparatus and a method for exactly detecting an occurrence of a crack in a probe in an early stage will be described.

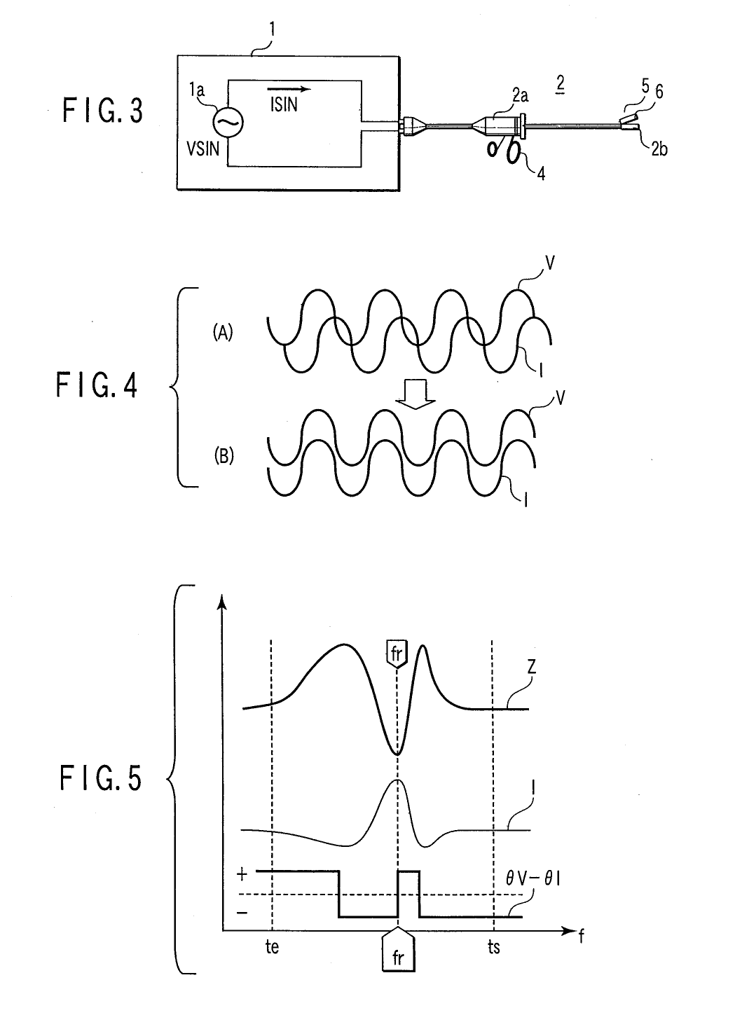

FIGS. 3 to 5 are views for explaining a method of controlling ultrasonic drive in an ultrasonic operation system. In FIG. 3, in an ultrasonic oscillator circuit 1a, a sinusoidal drive voltage VSIN is generated. When a sinusoidal drive current ISIN corresponding to the sinusoidal drive voltage VSIN flows into the ultrasonic vibrator inside the hand-piece main body section 2a, the ultrasonic vibrator converts the electric signal into mechanical vibration, and transmits the mechanical vibration to the distal end of the probe 2b. In the ultrasonic drive described above, when the ultrasonic vibration is output at a constant oscillation frequency, a phase difference occurs between the voltage V and the current I, and hence the drive efficiency lowers. Thus, a control circuit is provided in the ultrasonic power source unit 1, and the drive of the ultrasonic vibrator is performed while a resonance point at which a phase difference between the voltage V and the current I becomes 0 ((B) in FIG. 4) is searched for.

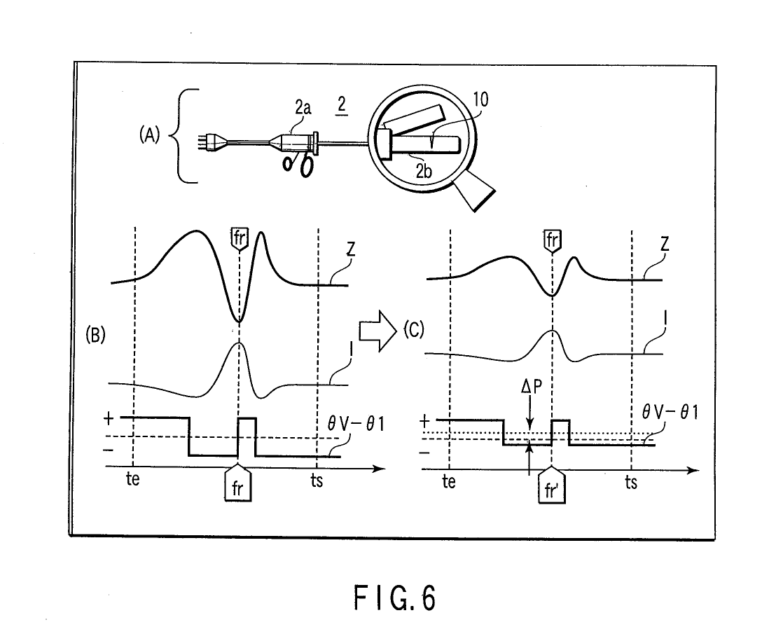

For example, in FIG. 5, the abscissa indicates the frequency f, and the ordinate indicates the impedance Z, current I, and phase difference (θV−θI). A value (θV−θI) indicates a phase difference. In this embodiment, a resonant frequency fr at which the phase difference (θV−θI) becomes 0 is detected by scanning for a point at which the impedance Z is minimized while consecutively changing the frequency. The control circuit 1c starts to perform the drive of the ultrasonic vibrator at the detected resonant frequency fr.

### FIRST EMBODIMENT

(A) to (C) in FIG. 6 are views for explaining a method of investigating an abnormality of a hand-piece 2 according to a first embodiment. (A) in FIG. 6 is a view showing a probe 2b part of the hand-piece 2 in an enlarging manner. This view schematically shows a state where the probe 2b has a crack 10. Here, the term crack does not necessarily imply a crack that can be confirmed with the naked eye, and includes a crack that does not appear externally, such as an internal crack, and a microcrack that appears at the early stage of metal fatigue. In the actual crack measurement, not only megascopic observation, but also microscopic observation using a magnifying glass, a metallurgical microscope or the like, and observation of a crack (microcrack) in the order of microns using an electron microscope are performed.

Measurement was conducted in detail so as to observe what variation occurs in the impedance Z and the phase difference (θV−θI) until a normal probe is cracked. The results are shown below.

(B) and (C) in FIG. 6 are graphs showing frequency dependence of the impedance Z, current I, and phase difference (θV−θI) which are under the PLL control observed when a crack has developed in the probe 2b in the normal state. At (B) in FIG. 6, the probe is not yet damaged, and the impedance Z, current I, and phase difference (θV−θI) which are in the normal state are shown. The frequency is varied by the PLL control such that the phase difference (θV−θI) becomes zero degree. At (B) in FIG. 6, the phase difference (θV−θI) becomes also zero degree in the vicinity of a frequency at which the impedance Z becomes the lowest. Accordingly, this frequency fr is the resonant frequency.

(C) in FIG. 6 shows a graph of the impedance Z, current I, and phase difference (θV-θI) under the PLL control observed after the probe 2b is cracked. When the crack develops in the probe 2b, it is conceivable that the phase difference (θV−θI) is shifted, and the impedance is also largely varied. Further, the PLL control is performed such that the impedance becomes the minimum, and a new resonant frequency fr′ is searched for. (C) in FIG. 6 shows the impedance Z, current I, and phase difference (θV−θI) observed after the search, and it can be seen that the control is performed such that the phase difference (θV−θI) becomes in the vicinity of zero at the new resonant frequency fr′. However, it can also be seen that the minimum value of the impedance Z is larger than that at (B) in FIG. 6, and the value of the phase difference (θV−θI) is also at a value (dotted line) higher than the zero value (broken line) before the occurrence of the crack by ΔP. In the illustration of the phase difference (θV−θI) shown at (B) and (C) in FIG. 6, the degree of the positive/negative magnitude, and the polarities are shown schematically and rectangularly only for easy understanding. The characters ΔP indicating the variation in the phase difference (θV−θI) can also be produced by factors other than the crack in the probe. However, the value is several degrees or less, and a variation exceeding 10 degrees is attributable to a crack.

Even when the PLL control is performed, the impedance Z is varied by the crack produced in the probe 2b. It is conceivable that the impedance of the entire probe 2b has been varied, whereby the frequency characteristic of the impedance has been varied, and the frequency dependence of the phase difference (θV−θI) between the current and the voltage has also been varied. More specifically, the reason why the value of the phase difference (θV−θI) exhibits a value higher than before by ΔP can be conceivable that the probe 2b cannot sufficiently exhibit the function of the probe serving as a complete vibration transmitting element of the ultrasonic vibrator due to the crack, and another interference mode resulting from the crack is mixed with the vibration.

On the basis of these results, and by paying attention to the impedance Z of the hand-piece 2 under the PLL control, it is possible to measure the fact that a crack has been produced in the probe 2b by monitoring the variation with time in the phase difference (θV−θI) between a voltage phase signal OV and a current phase signal θI.

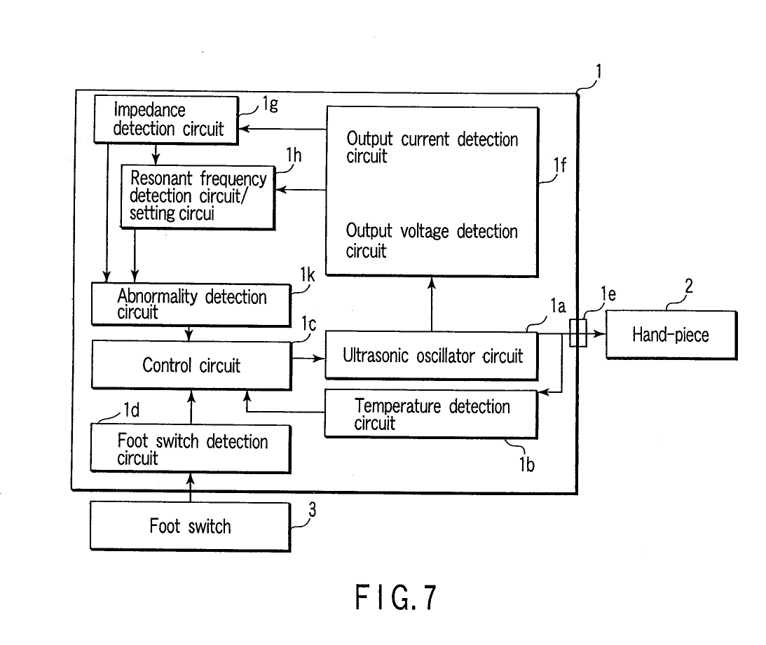

FIG. 7 is a functional block diagram for explaining a function of each unit in an ultrasonic power source unit in an ultrasonic operation system. The hand-piece 2 is connected to the ultrasonic power source unit 1 through a connector 1e. In the ultrasonic power source unit 1, an ultrasonic oscillator circuit 1a, output voltage/output current detection circuit 1f, impedance detection circuit 1g, resonant frequency detection circuit/setting circuit 1h, temperature detection circuit 1b, foot switch detection circuit 1d, and control circuit 1c are provided. The ultrasonic oscillator circuit 1a is a part for generating a drive signal for driving the ultrasonic vibrator inside the hand-piece 2. The foot switch detection circuit 1d is a part for detecting that the foot switch 3 has been operated by the operator.

When the foot switch 3 is operated by the operator, the operation signal is transmitted to the control circuit 1c through the foot switch detection circuit 1d. The control circuit 1c performs control such that the ultrasonic power is output from the ultrasonic oscillator circuit 1a to the hand-piece 2.

The output voltage/output current detection circuit 1f is a part for detecting an output voltage and an output current of the power supplied from the ultrasonic oscillator circuit 1a to the ultrasonic vibrator. The values of the output voltage and the output current detected by the output voltage/output current detection circuit 1f are input to the impedance detection circuit 1g and the resonant frequency detection circuit 1h. The impedance detection circuit 1g detects the impedance by using the impedance detection algorithm of the hand-piece 2 on the basis of the values of the input output voltage and the input output current, and the phase difference between them.

The resonant frequency detection circuit/setting circuit 1h detects a frequency actually applied to the probe 2b from the output voltage and the output current detected by the output voltage/output current detection circuit 1f, and at the same time, monitors a variation in the impedance value transmitted from the impedance detection circuit 1g. A frequency at which the value of the impedance abruptly changes is obtained, and detected as the resonant frequency. Further, the resonant frequency setting circuit 1h sets an allowable numerical range (defined as a predetermined numerical range) of the resonant frequency, and a variation amount (defined as a reference variation amount) allowable for a variation in the resonant frequency per unit time.

The abnormality detection circuit 1k chronologically stores the value of the resonant frequency transmitted from the resonant frequency detection circuit/setting circuit 1h, the predetermined numerical range, and the variation amount of the resonant frequency in the internal storage part. More specifically, the value of the resonant frequency is saved in a memory which is the storage part at intervals of, for example, 5 msec, and the consecutively measured value of the resonant frequency and the previously saved value of the resonant frequency are compared with each other, and it is monitored whether or not the resonant frequency is within the predetermined numerical range. Further, the value of the impedance measured at intervals of 5 msec is compared with plural values of the resonant frequency such as values measured 5 msec ago, 10 msec ago, 15 msec ago, and so on, thereby judging whether or not the variation in the value of the resonant frequency is not abnormal as compared with the reference variation amount. For example, a reference variation amount to be set by the resonant frequency setting circuit may be set with respect to the variation amount of the resonant frequency per unit time, and the set reference variation amount may be transmitted to the abnormality detection circuit 1k. The abnormality detection circuit 1k subjects the value of the resonant frequency and the variation amount per unit time transmitted from the resonant frequency detection circuit/setting circuit 1h to calculation, compares the calculation results with the predetermined numerical range and the reference variation amount which have been transmitted from the circuit 1h, and judges that the state of the resonant frequency is abnormal when the calculation results exceed the predetermined numerical range and the reference variation amount.

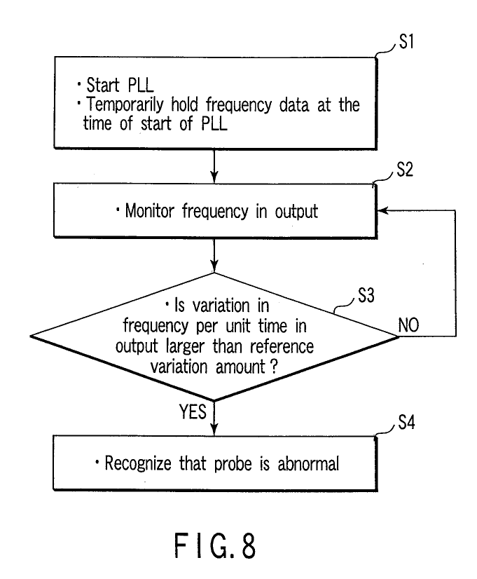

The above flow will be described below by using the flowchart of FIG. 8. First, when an operation in an abdominal cavity of a patient is performed by using an ultrasonic probe 2b, the control circuit 1c starts the PLL control, and the abnormality detection circuit 1k detects the initial resonant frequency, and stores the detected data (step S1). The PLL control is the control necessary for the ultrasonic probe to perform an operation with increased energy efficiency. While the ultrasonic power is output from the ultrasonic oscillator circuit 1a to the hand-piece 2, the abnormality detection circuit 1k monitors the variation in the resonant frequency at intervals of a fixed sampling time determined in advance (step S2). The monitored resonant frequency is compared with a plurality of resonant frequency data items detected previously. For example, the abnormality detection circuit 1k determines to set the sampling time at 5 msec, and compares each of 20 samples of the resonant frequency (resonant frequency values within a period of 5 msec×20 samples 100 msec) detected previously, or an average value of the 20 samples of the resonant frequency detected previously with a currently detected resonant frequency. The abnormality detection circuit 1k compares a variation in the resonant frequency per unit time (100 msec) with the reference variation amount, for example, 500 Hz/100 msec (step S3), and judges that the probe is abnormal when the variation is larger than the reference variation amount (step S4). When the variation is smaller than the reference variation amount, the abnormality detection circuit 1k judges that the probe 2b is normal, and returns to step S2 to continue monitoring the variation in the resonant frequency.

A correlation between the actually measured value of the resonant frequency and the crack occurrence status of the probe 2b was measured. As a result of the measurement, when the variation in the resonant frequency exceeds 500 Hz, a crack that can be visually confirmed, or a microcrack that can be confirmed by using an electron microscope occurred.

### (Effect)

According to this embodiment, the resonant frequency is detected, the resonant frequency variation amount per unit time of the resonant frequency is monitored, and a resonant frequency variation amount different from a resonant frequency variation amount resulting from resection or the like of living tissue by an ordinary operation is detected as an abnormality, whereby it is possible to instantaneously and easily grasp an occurrence of a crack in the probe. By virtue of the detection of the probe crack in the early stage, the medical staff can replace the probe before the breakage of the probe occurs, and safely continue the treatment of the patient.

### Second Embodiment

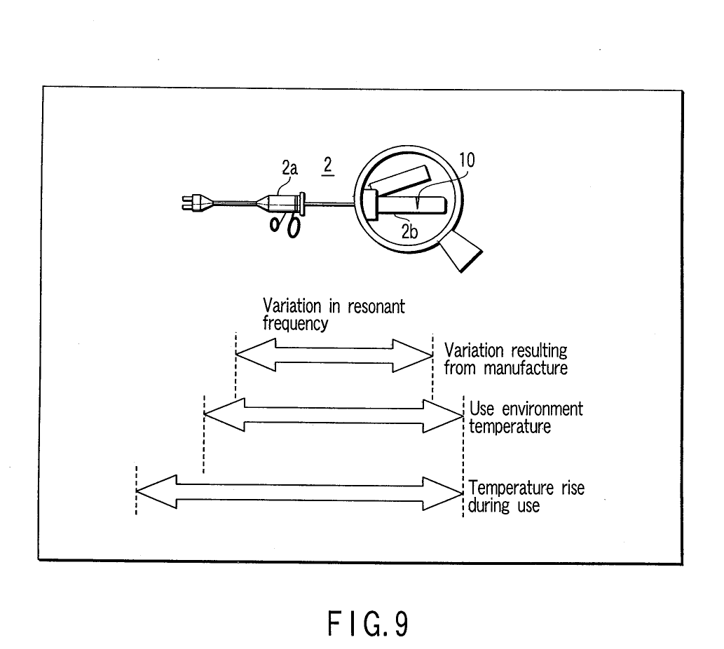

A second embodiment of the present invention will be described below. Here, how to determine the reference variation amount will be described below. FIG. 9 shows the magnitude of each factor in the causation of a variation in a resonant frequency by the size of the arrow. Among the variations in the resonant frequency, the variation resulting from a crack 10 of the probe 2b is the largest. However, as factors other than the crack, the variation in the product resulting from the manufacture, use environment temperature, and temperature rise during use which become larger in the order mentioned are present. Particularly, the temperature rise during use is due to the output of power to the ultrasonic vibrator. The temperature rise during use differs depending on the type of the ultrasonic vibrator. In a certain type of ultrasonic vibrator, a temperature rise of +10° C. is observed during use, and in another type of ultrasonic vibrator, a temperature rise of +30° C. is observed. Because of the rise in temperature in these ultrasonic vibrators, a variation in the resonant frequency from about 300 to 400 Hz is observed. A correlation between the temperature rise of the ultrasonic vibrator and the variation in the resonant frequency can be measured in advance. Further, it is also known that the temperature of the ultrasonic vibrator is well correlated with the electric capacitance of the ultrasonic vibrator. Accordingly, the temperature of the ultrasonic vibrator can be obtained with high accuracy by measuring, for example, the electric capacitance of the ultrasonic vibrator, and the variation amount of the resonant frequency can also be estimated on the basis of the temperature.

More specifically, the temperature can be measured by measuring, on the basis of the fact that the electric capacitance of the hand-piece 2 in which the ultrasonic vibrator is incorporated is correlated with the internal temperature thereof, the electric capacitance. Accordingly, a variation amount of the resonant frequency is compared with the variation amount of the resonant frequency resulting from the temperature, and when it is judged that the variation amount of the resonant frequency is an amount larger than the variation amount of the resonant frequency resulting from the temperature, the probe is judged to be abnormal, the ultrasonic output is stopped or shut down. As described above, the abnormality detection circuit 1k defines the variation amount corresponding to the detected temperature as the reference variation amount, and performs detection to confirm whether or not the variation is within the range.

### (Effect)

It is effective to set a variation in the resonant frequency resulting from the temperature of the ultrasonic vibrator as a reference variation amount with respect to the variation amount of the resonant frequency per unit time. By this setting method of the reference variation amount, it is possible to accurately and easily distinguish a change in the resonant frequency resulting from the temperature rise at the time of an ordinary operation and a change in the resonant frequency resulting from a crack of the probe 2b from each other. In accordance with the above, it is possible to stop or shut down the ultrasonic output, and prevent breakage or falling off of the probe greater than the crack.

### Third Embodiment

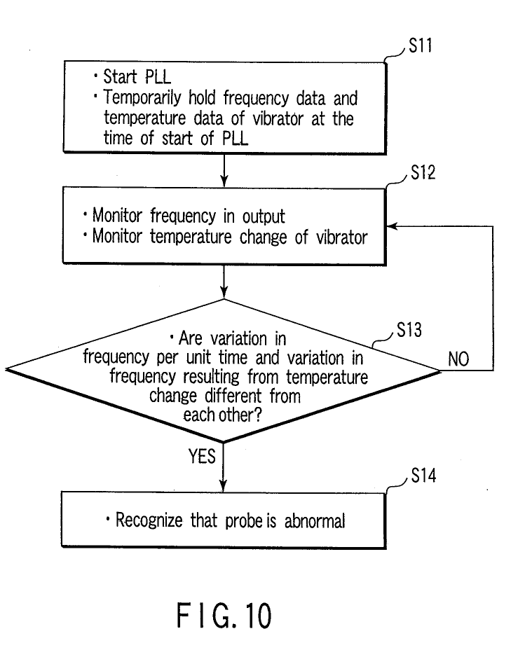

A third embodiment of the present invention will be described below by using the block diagram of FIG. 7 and the flowchart of FIG. 10. First, when an operation in an abdominal cavity of a patient is performed by using an ultrasonic probe 2b, the control circuit 1c starts the PLL control, and the abnormality detection circuit 1k detects the initial resonant frequency, stores the detected data, detects the temperature of a hand-piece 2 in which an ultrasonic vibrator is incorporated by means of a temperature detection circuit 1b, and stores the detected data (step S11). Actually, the temperature detection circuit 1b measures the electric capacitance of the hand-piece 2, and calculates the temperature of the hand-piece 2 by using a correlation formula of the temperature and the electric capacitance measured in advance. While the ultrasonic power is output from the ultrasonic oscillator circuit 1a to the hand-piece 2, the abnormality detection circuit 1k monitors the variation in the resonant frequency at intervals of a fixed sampling time determined in advance, and simultaneously monitors the temperature of the hand-piece 2 (step S12). As for the unit time, for example, the sampling time is set at 5 msec. The abnormality detection circuit 1k judges whether or not the variation in the resonant frequency per unit time and the variation in the resonant frequency resulting from the temperature change are different from each other (step S13). At this time, a step of setting a variation amount (reference variation amount) of the resonant frequency per unit time resulting from the change in temperature may be provided in advance in the resonant frequency setting circuit 1h, and the set variation amount may be transmitted to the abnormality detection circuit 1k. When the actual variation in the resonant frequency is larger than the variation in the resonant frequency resulting from the temperature, the abnormality detection circuit 1k judges that the probe is abnormal (step S14). When the actual variation in the resonant frequency is identical with the variation in the resonant frequency resulting from the temperature, the abnormality detection circuit 1k judges that the probe 2b is normal, and returns to step S12 to continue monitoring the variation in the resonant frequency.

When the actually measured variation in the resonant frequency exceeds the variation in the resonant frequency resulting from the change in temperature, the occurrence status of the crack of the probe 2b was investigated. As a result, when the variation in the resonant frequency exceeds the variation in the resonant frequency resulting from the change in temperature, a crack that can be visually confirmed or a microcrack that can be confirmed by using an electron microscope occurred.

### (Effect)

When the variation in the resonant frequency is larger than the reference variation amount, the probe is judged to be abnormal. By the judgment of the abnormality, a more accurate and appropriate judgment is made, and the ultrasonic output is stopped or shut down.

### Fourth Embodiment

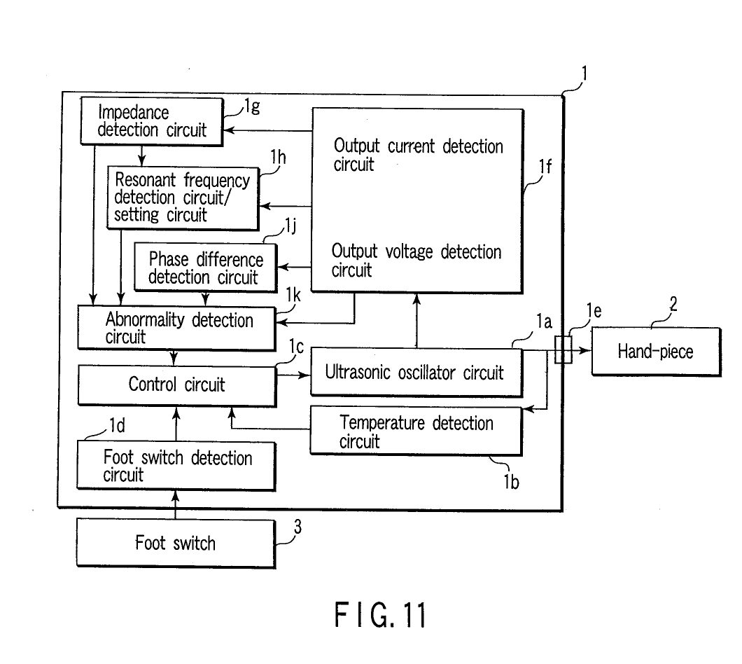

A fourth embodiment will be described below with reference to the block diagram of FIG. 11. This block diagram resembles the block diagram of FIG. 7, and includes a phase difference detection circuit 1j in addition to the block diagram of FIG. 7. It is known from (B) and (C) in FIG. 6 that a phase difference (θV−θI) between an output voltage and an output current which are detected by the phase difference detection circuit 1j is varied by a crack of the probe 2b. The variation in the phase difference can further be used as the abnormality judgment means. Further, an abnormality detection circuit 1k acquires signals of the output voltage and the output current from an output voltage/output current detection circuit 1f. It is known from (B) and (C) in FIG. 6 that the output current or the like is also varied by the crack of the probe 2b. Accordingly, the variation in the output current or the like can further be used as the abnormality judgment means.

### (Effect)

By measuring a variation amount of the phase difference (θV−θI), the output current, or the like, a crack of the probe can be grasped more accurately and appropriately.

### Fifth Embodiment

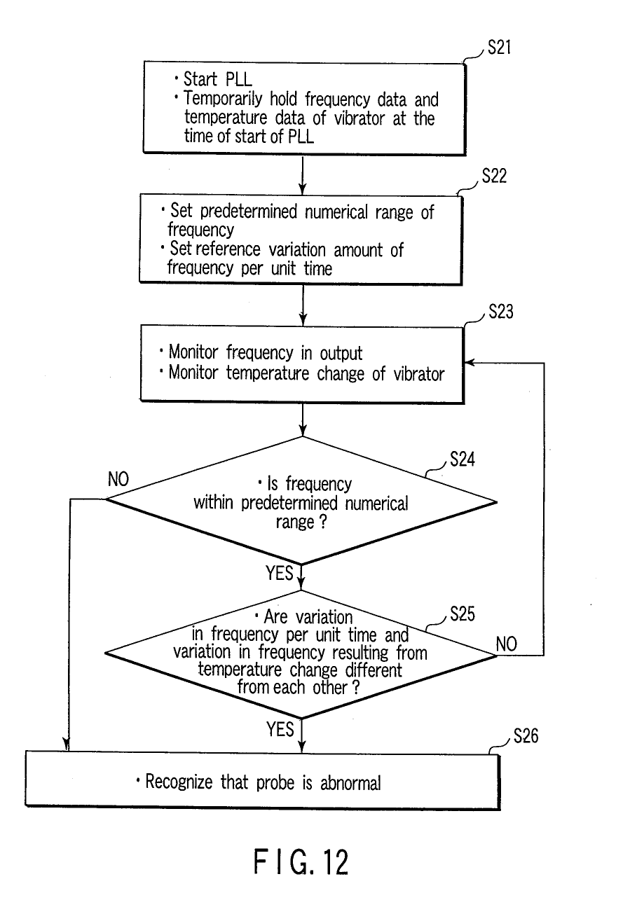

A fifth embodiment of the present invention will be described below by using the block diagram of FIG. 11 and the flowchart of FIG. 12. First, when an operation in an abdominal cavity of a patient is performed by using an ultrasonic probe 2b, the control circuit 1c starts the PLL control, and the abnormality detection circuit 1k detects the initial resonant frequency, stores the detected data, detects the temperature of a hand-piece 2 in which an ultrasonic vibrator is incorporated by means of a temperature detection circuit 1b, and stores the detected data (step S21). Actually, the temperature detection circuit 1b measures the electric capacitance of the hand-piece 2, and calculates the temperature of the hand-piece 2 by using a correlation formula of the temperature and the electric capacitance measured in advance. A resonant frequency setting circuit 1h can set an allowable predetermined numerical range of the resonant frequency, and an allowable variation amount (defined as a reference variation amount) of the resonant frequency per unit time automatically or manually (step S22). When manual setting is performed, an external input terminal (not shown) is used to directly input the data to the resonant frequency setting circuit 1h. As a method for automatically inputting the data, a variation amount of the resonant frequency per unit time resulting from the temperature change of the surgical instrument is measured in advance, and the numerical range and the variation amount of the resonant frequency can be automatically calculated at any time from the temperature of the surgical instrument detected on the basis of the measured data. Further, the set numerical range and the variation amount are transmitted to the abnormality detection circuit 1k. Although this resonant frequency setting circuit is arranged in the block diagram of FIG. 11 as the circuit 1h together with the resonant frequency detection circuit, it may be incorporated in the abnormality detection circuit 1k. While the ultrasonic power is output from the ultrasonic oscillator circuit 1a to the hand-piece 2, the abnormality detection circuit 1k monitors the variation in the resonant frequency at intervals of a fixed sampling time determined in advance, and simultaneously monitors the temperature of the hand-piece 2 (step S23). As for the unit time, for example, the sampling time is set at 5 msec. The abnormality detection circuit 1k detects whether or not the value of the resonant frequency is within the predetermined numerical range (step S24). When the value of the resonant frequency is within the predetermined numerical range, the probe is judged to be normal, and the flow advances to next step S25. When the value of the resonant frequency is not within the predetermined numerical range, the probe is judged to be abnormal (step S26).

When it is judged in step S24 that the probe is normal, then the abnormality detection circuit 1k judges whether or not the variation amount of the resonant frequency per unit time and the variation amount (reference variation amount) of the resonant frequency resulting from the temperature change are different from each other (step S25). When the amount of the actual variation in the resonant frequency is larger than the variation amount of the resonant frequency resulting from the temperature change, the abnormality detection circuit 1k judges that the probe is abnormal (step S26). When the amount of the actual variation in the resonant frequency is identical with the variation amount of the resonant frequency resulting from the temperature change, the abnormality detection circuit 1k judges that the probe is normal, and returns to step S23 to continue monitoring the variation in the resonant frequency.

As specific numerical values, the cases of two surgical instruments (HP1 and HP2) will be described. In the case of HP1, the predetermined numerical range of the resonant frequency is set as a range of 46.5 kHz to 47.5 kHz, and the reference variation amount is set at 0.2 kHz. In the case of HP2, the predetermined numerical range of the resonant frequency is set as a range of 46.3 kHz to 47.7 kHz, and the reference variation amount is set at 0.12 kHz. In the case of each of the surgical instruments, when the actual resonant frequency or the variation amount of the resonant frequency deviated from or exceeded the set predetermined numerical range or the reference variation amount, the occurrence status of the crack of the probe 2b was investigated. As a result, when the value of the resonant frequency or the variation amount of the resonant frequency deviated from or exceeded the predetermined numerical range or the reference variation amount, a crack that could be visually confirmed or a microcrack that could be confirmed by using an electron microscope occurred.

### (Effect)

When the value of the resonant frequency or the variation amount of the resonant frequency deviates from or exceeds the predetermined numerical range or the reference variation amount, the probe is judged to be abnormal. By the judgment of the abnormality, a more accurate and appropriate judgment is made, and the ultrasonic output is stopped or shut down.

### Sixth Embodiment

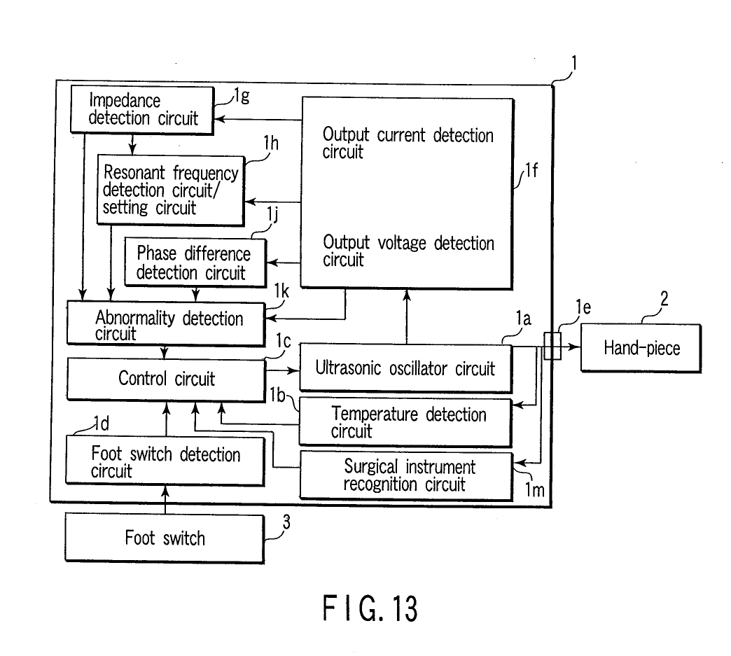

A sixth embodiment will be described below with reference to the block diagram of FIG. 13. This block diagram resembles the block diagram of FIG. 11, and includes a surgical instrument recognition circuit 1m in addition to the block diagram of FIG. 11. The surgical instrument recognition circuit 1m is the means for recognizing the type of a connected surgical instrument, for example, the type of a hand-piece. The resonant frequency characteristics of the surgical instruments discriminated from each other by the surgical instrument discrimination circuit 1m are measured in advance with respect to the temperature change according to the type of the instruments. On the basis of the resonant frequency characteristic of the surgical instrument measured with respect to the temperature change, the predetermined numerical range and the reference variation amount can be set in advance.

### (Effect)

By using the surgical instrument recognition circuit 1m, even when different surgical instruments are provided, it is possible to accurately set the predetermined numerical range and the reference variation amount, and grasp the crack of the probe more accurately and appropriately.

What is claimed is:

1. A power supply apparatus for operation for outputting power to a surgical instrument comprising:
a resonant frequency detection section for detecting a resonant frequency which minimizes the impedance of the surgical instrument;
a resonant frequency setting section for setting in advance an allowable variation amount of the resonant frequency per unit time as a reference variation amount; and
an abnormality detection section for detecting whether or not a detected variation amount of the resonant frequency per unit time exceeds the reference variation amount.

2. The power supply apparatus for operation according to claim 1, further comprising a temperature detection section for detecting a temperature of the surgical instrument, wherein the reference variation amount is a variation amount of the resonant frequency which varies in accordance with an amount of change in the temperature of the surgical instrument detected by the temperature detection section.

3. The power supply apparatus for operation according to claim 2, wherein
in the resonant frequency setting section, an allowable predetermined numerical range of the resonant frequency is further set, and
the abnormality detection section further detects whether or not the resonant frequency detected by the resonant frequency detection section is within the predetermined numerical range.

4. The power supply apparatus for operation according to claim 3, wherein the abnormality detection section detects whether or not the detected resonant frequency is within the predetermined numerical range of the resonant frequency corresponding to the temperature of the surgical instrument detected in advance.

5. The power supply apparatus for operation according to claim 4, further comprising a surgical instrument recognition section for recognizing the type of a connected surgical instrument, wherein
the abnormality detection section detects whether or not the resonant frequency detected by the resonant frequency detection section is within the predetermined numerical range corresponding to the surgical instrument recognized by the surgical instrument recognition section.

6. The power supply apparatus for operation according to claim 5, wherein when the detected variation amount of the resonant frequency per unit time exceeds the reference variation amount, or when the detected resonant frequency is not within the predetermined numerical range corresponding to the temperature and the type of the surgical instrument, the abnormality detection section stops the power supply to the surgical instrument.

7. The power supply apparatus for operation according to claim 3, wherein the abnormality detection section detects whether or not the detected resonant frequency is within the predetermined numerical range corresponding to the predetermined temperature change of the surgical instrument.
